(19) United States
(12) Patent Application Publication
(10) Pub. No.: US 2026/0345953 A1
(43) Pub. Date: Nov. 5, 2026

(54) LEAKAGE-RESISTANT CATHETER ASSEMBLY

(71) Applicant: Lars Medicare Private Limited, Sonipat (IN)

(72) Inventor: Surmya Goel, New Delhi (IN)

(73) Assignee: Lars Medicare Private Limited, Sonipat (IN)

(21) Appl. No.: 19/332,911

(22) Filed: Sep. 18, 2025

(30) Foreign Application Priority Data
Apr. 22, 2025 (IN) 202511038760

(51) Int. Cl. A61M 25/00; A61M 25/06; A61M 39/24

(52) U.S. Cl. CPC A61M 25/0023; A61M 25/0097; A61M 25/065; A61M 39/24; A61M 2039/2426

(57) ABSTRACT

An embodiment herein provides a catheter assembly for controlling fluid leakage. The catheter assembly includes a catheter adapter with a lumen defined within the catheter adapter. The catheter assembly includes a catheter extending distally from a distal end of the catheter adapter. The catheter assembly includes a fluid control valve positioned within the lumen of the catheter adapter. The fluid control valve includes a plus-shaped slit adapted to selectively allow passage of a needle therethrough. The catheter assembly includes an actuator deployed within the catheter adapter and having a generally circular cross-sectional profile. The actuator is sized to engage with the plus-shaped slit of the fluid control valve.

### CROSS-REFERENCE TO RELATED APPLICATIONS

This application claims priority to Indian Patent Application No. 202511038760, filed on Apr. 22, 2025, the disclosure of which is incorporated herein by reference in its entirety.

### BACKGROUND

### Technical Field

The embodiments herein generally relate to intravenous (IV) catheter assemblies, and in particular, to catheter assemblies with integrated fluid control mechanisms for preventing fluid leakage.

### Description of the Related Art

Intravenous (IV) catheter assemblies are widely used in clinical and emergency settings to provide vascular access for administration of fluids, medications, or for blood sampling. These devices typically include a catheter connected to a hub or adapter through which a needle is inserted to gain venous access. Once access is achieved, the needle is withdrawn, leaving the catheter in place for continued fluid delivery or sampling.

A major challenge associated with conventional catheter assemblies is the risk of fluid leakage such as blood after needle removal. To mitigate this, fluid control valves have been introduced, which close automatically upon needle withdrawal to seal the fluid path. However, many existing valve designs either compromise flow characteristics, are prone to wear and tear, or require complex actuation mechanisms.

There is therefore a need for an improved catheter assembly that provides reliable fluid sealing, is simple in construction, and maintains optimal performance over repeated use.

### SUMMARY

In an embodiment, a catheter assembly is provided for controlling fluid leakage. The catheter assembly includes a catheter adapter having a distal end, a proximal end, a body portion extending between the distal end and the proximal end, and a lumen defined within the catheter adapter. The lumen extends through the body portion between the distal end and the proximal end. The catheter assembly includes a catheter extending distally from the distal end of the catheter adapter, such that the catheter is in fluid communication with the lumen. The catheter assembly includes a fluid control valve positioned within the lumen of the catheter adapter, the fluid control valve comprising a plus-shaped slit adapted to selectively allow passage of a needle therethrough. The catheter assembly includes an actuator deployed within the catheter hub and having a generally circular cross-sectional profile. The actuator includes a proximal end and a distal end, wherein the distal end is sized to engage with the plus-shaped slit of the fluid control valve and the proximal end has a larger diameter than the distal end. The actuator tapers from the distal end to the proximal end at a predefined angle.

In an embodiment, a catheter assembly is provided for controlling fluid leakage. The catheter assembly includes a catheter adapter having a distal end, a proximal end, a body portion extending between the distal end and the proximal end, and a lumen extending through the body portion. The catheter assembly includes a catheter extending distally from the distal end of the catheter adapter and in fluid communication with the lumen. The catheter assembly includes a fluid control valve disposed within the lumen of the catheter adapter. The fluid control valve includes a plus-shaped slit configured to selectively permit passage of a needle therethrough. The catheter assembly includes an actuator positioned within the catheter adapter and having a proximal end and a distal end. The distal end is dimensioned to engage the plus-shaped slit of the fluid control valve. The proximal end has a diameter larger than the distal end. The actuator tapers from the distal end toward the proximal end at a predefined angle, wherein engagement of a luer connector with the actuator urges the actuator distally to open the fluid control valve and reduce fluid leakage following withdrawal of the needle.

In some embodiments, a catheter assembly is provided for fluid delivery. The catheter assembly includes a catheter adapter defining a lumen extending between a proximal end and a distal end. The catheter assembly includes a catheter extending from the distal end of the catheter adapter and in fluid communication with the lumen. The catheter assembly includes a valve disposed within the lumen and including a slit adapted to permit passage of a needle and to substantially reseal following removal of the needle. The catheter assembly includes an actuator disposed within the catheter adapter and engageable with the slit of the valve to facilitate controlled opening and closing of the valve during coupling with a connector.

### BRIEF DESCRIPTION OF THE DRAWINGS

The features of the disclosed embodiments may become apparent from the following detailed description taken in conjunction with the accompanying drawings showing illustrative embodiments herein, in which:

FIG. 1 is a cross-sectional view of a catheter assembly for controlling fluid leakage, illustrating an internal structure and spatial relationship of various components of the catheter assembly, in accordance with an embodiment.

FIG. 2 is a top view of the catheter assembly of FIG. 1, in accordance with some embodiments.

FIG. 3 is a front view of the catheter assembly of FIG. 1, in accordance with some embodiments.

FIG. 4 is a side left view of the catheter assembly of FIG. 1, in accordance with some embodiments.

FIG. 5 is a side right view of the catheter assembly of FIG. 1, in accordance with some embodiments.

FIG. 6 is a perspective view of the catheter assembly of FIG. 1, in accordance with some embodiments.

FIG. 7 illustrates a top view of a fluid control valve of the catheter assembly, in accordance with some embodiments.

FIG. 8 illustrates a perspective view of the catheter assembly with a luer connector, in accordance with some embodiments.

### DETAILED DESCRIPTION

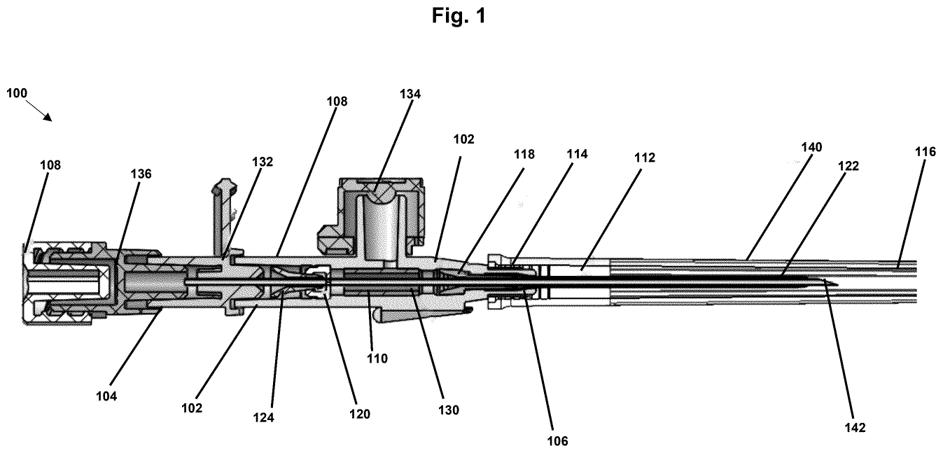

FIG. 1 is a cross-sectional view of a catheter assembly 100 for controlling fluid leakage, illustrating an internal structure and spatial relationship of various components of the catheter assembly 100, in accordance with an embodiment of the present disclosure.

The catheter assembly 100 includes a catheter adapter 102 having a proximal end 104, a distal end 106, and a body portion 108 extending therebetween. The catheter adapter 102 includes a lumen 110 that is defined within the catheter adapter 102 and extends longitudinally from the proximal end 104 to the distal end 106. The catheter assembly 100 further includes a catheter 112 with a proximal end 114 and a distal end 116. The catheter 112 may be fixedly secured to and extends distally from the distal end 106 of the catheter adapter 102. The catheter 112 is in fluid communication with the lumen 110 of the catheter adapter 102. The catheter 112 may be supported by a Teflon holder 118 that may be disposed around the catheter 112 at its proximal end or region 114. The Teflon holder 118 acts as a hinge and is configured to provide a supporting structure between the catheter 112 and the catheter adapter 102 that holds the catheter 112 in place relative to the catheter adapter 102.

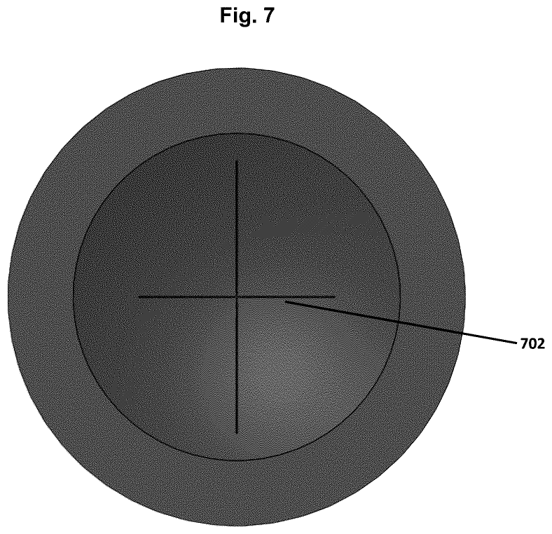

The catheter assembly includes a fluid control valve 120 that is positioned coaxially within the lumen 110 of the catheter adapter 102. The fluid control valve 120 comprises a plus-shaped slit 702 (shown in FIG. 7) adapted to permit selective passage of a needle 122 through the fluid control valve 120 while restricting fluid flow when the needle 122 is removed. The fluid control valve 120 may be secured within an annular groove inside the catheter adapter 102 such that axial movement in either a proximal or distal direction is restricted.

The catheter assembly 100 includes an actuator 124 that may be positioned proximally relative to the fluid control valve 120. The actuator 124 may have a generally circular cross-sectional profile and comprises a distal end 126 sized to engage with the plus-shaped slit 702 of the fluid control valve 120, and a proximal end 128 having a larger diameter. The actuator 124 tapers from the distal end 126 to the proximal end 128 at a predefined angle, which facilitates forward movement and interaction with a luer lock connector to open the fluid control valve during insertion, and to re-seal upon withdrawal of the needle 122. The actuator 124 may be provided with external features to interact with corresponding stop structures within the catheter adapter 102, preventing over-advancement.

In some embodiments, proximally adjacent to the actuator 124 is a tube 130 which is configured to allow fluid to pass easily toward the catheter smoothly without causing back pressure. The tube 130 is positioned proximally relative to the catheter adapter 102 and is operably connected to a needle hub 132. The needle hub 132 is provided to be configured to receive the proximal portion of the needle 122 and connects to an upstream infusion or injection device. In an embodiment, a port cap 134 may be disposed atop the catheter adapter 102, providing access to internal components for assembly or sterilization purposes. In some other embodiments, the catheter adapter 102 may not have the port cap 134. In some embodiments, the catheter assembly 100 may have the tube 130. In some embodiments, the catheter assembly 100 may not have the tube 130.

A hub cover 136 may be provided to surround the proximal end or region 104 of the catheter adapter 102 and encases fluid pathway. A thread stopper 138 may be disposed at a proximal-most end of the catheter assembly 100 and is configured to engage with one or more external luer lock connectors to provide a secure threaded coupling. The hub cover 136 and the thread stopper 138 may collectively form a luer-compatible interface. The hub cover 136 is the area where a user can see flashback of blood during needle 122 insertion and ensure proper deployment of the catheter 112 to a patient.

The needle 122 extends through a needle cover 140 positioned concentrically within the catheter 112 and terminates at a beveled distal tip 142 adapted for vascular penetration. The needle cover 140 may provide a protective guide to minimize damage to internal components during insertion and withdrawal of the needle 122.

The cross-sectional view of the catheter assembly 100 shown in FIG. 1 illustrates the internal structure and interconnection of each component within the catheter assembly 100, all configured to operate together to allow needle-based access while minimizing fluid leakage through action of the fluid control valve 120 and the actuator 124.

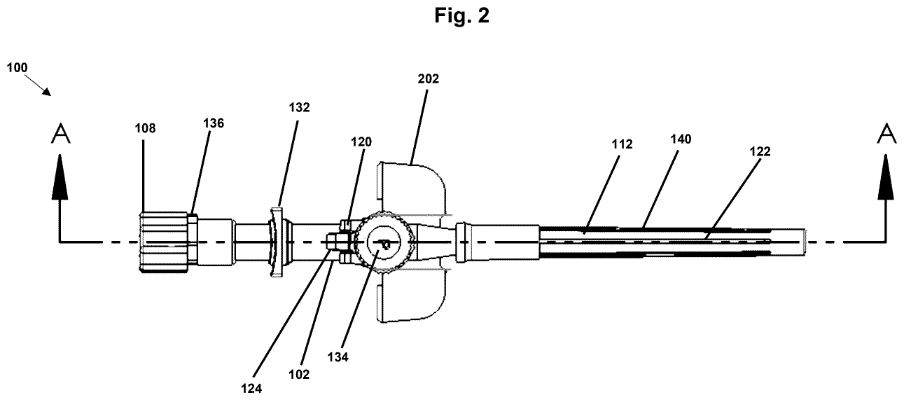

FIG. 2 is a top view of the catheter assembly of FIG. 1, illustrating the relative placement and alignment of the various components of the catheter assembly 100 in a different view. The catheter 102 is shown extending distally from the catheter adapter 102. The port cap 134 is disposed atop the catheter adapter 102. The actuator 124 and the fluid control valve 120 are disposed within the catheter adapter 102. In embodiments, a plurality of wings 202 are designed and provided on the catheter assembly 100 to facilitate secure grip and stabilization during insertion and maintenance. The wings 202 offer multiple gripping alternatives, allowing for easier manipulation and secure fixation to the skin, minimizing movement and preventing accidental dislodgement.

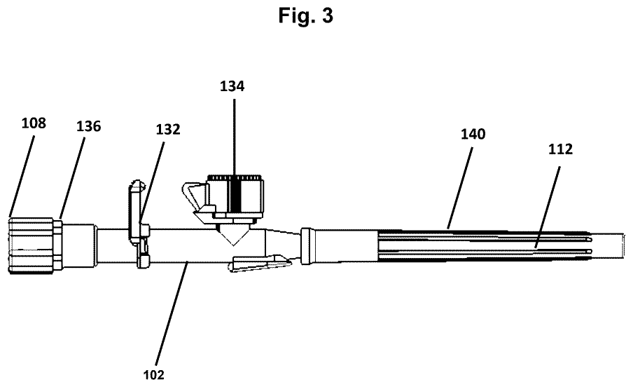

FIG. 3 is a front view of the catheter assembly of FIG. 1. The FIG. 3 depicts the various components of the catheter assembly 100 in a different view. Internal components such as the fluid control valve 120 and the actuator 124 are not visible in the front view.

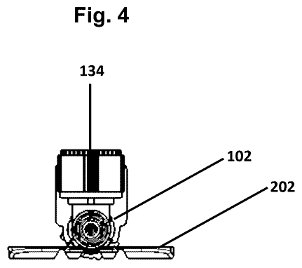

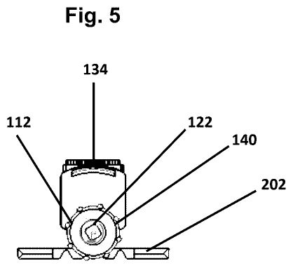

FIG. 4 is a side view (left) of the catheter assembly 100 of FIG. 1. The figure provides a side elevation profile of the catheter assembly 100. FIG. 5 is a side view (right) of the catheter assembly 100 of FIG. 1, further showing an opposite lateral projection of the catheter assembly 100.

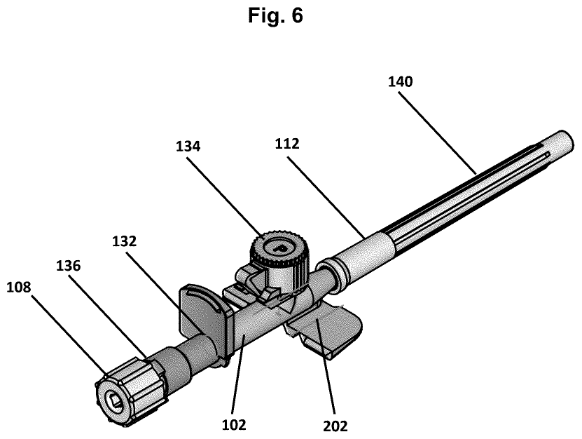

FIG. 6 is a perspective view of the catheter assembly of FIG. 1, illustrating three-dimensional relationship among the various structural components of the catheter assembly 100. The perspective view highlights geometry and external features of each component and their cooperative interface within the catheter assembly 100.

FIG. 6 illustrates a top view of the valve 120 showing the slit 702.

Referring to the FIGS. 1 through 7, the catheter assembly 100 is further described herein in some embodiments.

In various embodiments, the catheter assembly 100 as disclosed herein incorporates the fluid control valve 120 and the actuator 124 that function in tandem to substantially prevent fluid leakage when the needle 122 is withdrawn, while also ensuring ease of access during the needle 122 insertion. This design achieves this with minimal disruption to flow characteristics and provides structural adaptability for various clinical requirements.

The fluid control valve 120, positioned coaxially within the lumen 110 of the catheter adapter 102, may comprise a resilient membrane with the plus-shaped slit 702 (also referred to as slit 702 interchangeably without limitations throughout this document) formed therein. This slit 702 is centrally located and intersects in at least four directions, forming a symmetric or asymmetric cross pattern in various embodiments.

In various embodiments, the slit 702 may include a plurality of slit segments. In an embodiment, the slit segments may intersect orthogonally, creating a symmetrical, perpendicular cross. This configuration may allow for elastic deformation during the insertion of the needle 122 or the actuator 124 and self-retraction to a sealed state upon removal of the needle 122. The elasticity of the material, preferably a biocompatible elastomer such as silicone or thermoplastic elastomer (TPE), may provide dynamic sealing properties.

In an embodiment, the slit segments may diverge from its center at oblique angles (for example 45 °, 60 °, or 30°and the like relative to a central axis of the valve 120), forming an asymmetric or angled plus-shape or cross referred interchangeably without limitations. This angular arrangement may increase sealing pressure in certain orientations, particularly under pulsatile or pressurized flow, thereby enhancing leakage prevention of the fluid such as the blood.

In embodiments, the slit segments may be formed with variable widths. For example, a central crossing zone may have a narrower width, increasing elasticity at the center, while terminal ends of each slit segment may widen to better accommodate deformation during passage of the needle. This variation may help in controlled expansion and retraction behavior of the valve, particularly for needles with varying gauges.

In some embodiments, the valve 120 may include multiple layers, where an inner layer is composed of an elastic sealing material and an outer layer provides mechanical reinforcement. The outer layer may be a more rigid polymer or a mesh insert, reducing fatigue during repeated use. This dual-layered construction may improve wear resistance, dimensional stability, and valve lifespan.

An annular groove may be provided to anchor the valve within the catheter adapter 102, that may be configured to receive a peripheral flange or ridge on a body of the valve 120. This may restrict axial movement during connection or needle manipulation and provide a stable seal interface.

In some embodiments, the plus-shaped slit 702 may include micro-textures or surface ridges at its contact faces. These features may be designed to interface with corresponding features on the actuator 124, creating interlocking friction or pressure zones that inhibit unintentional valve 120 opening or fluid escape.

The actuator 124 is positioned proximally to the fluid control valve 120 and is designed to selectively engage with the slit 702 to momentarily open the valve during device connection or fluid administration. The actuator 124 has a generally circular cross-section and a body that tapers distally, reducing in diameter toward a region interfacing with the valve 120. This tapered profile of the adapter 124 is not merely aesthetic or space-saving; instead it is functionally significant. A predefined angle of tapering, typically in the range of 2% to 10% in various embodiments, facilitates gradual interaction with the slit 702, reducing mechanical stress and allowing a progressive seal disruption rather than abrupt rupture. This may minimize damage to edges of the slit 702 and ensure longevity of the structure or body of the valve 120. In accordance with the illustrated embodiments, the predefined angle is 6 degree.

In embodiments, the actuator 124 may include a stepped diameter design, where a distal region of the actuator 124 is narrow enough to just engage the slit 702 without forcing it open completely, while a proximal region of the adapter 124 is larger to ensure full opening when necessary (e.g., during luer lock connection). This differential geometry may allow for precision control over valve opening depending on insertion depth.

In some embodiments, the actuator 124 may include external micro-ridges or helical threads along its length. These features may interface with internal wings or stops within the catheter adapter 102, which act as mechanical brakes to restrict over-insertion of the actuator. Such control is necessary to avoid damaging the valve 120 or applying excessive pressure that could distort the fluid path.

In some other embodiments, engagement features such as key-and-slot structures or radial tabs may be incorporated to align the actuator 124 with the plus-shaped slit 702. This may help the actuator 124 engage symmetrically, providing even force distribution across all four slit segments.

In embodiments, the actuator 124 may include locking grooves or click-stop features that may align with complementary parts of the catheter adapter 102. These enable snap-fit retention, maintaining the actuator 124 in a stable state when the needle 122 is removed but before the luer connector is attached.

Similarly, in various embodiments, other mechanisms may be deployed without limiting the scope and spirit of the present invention.

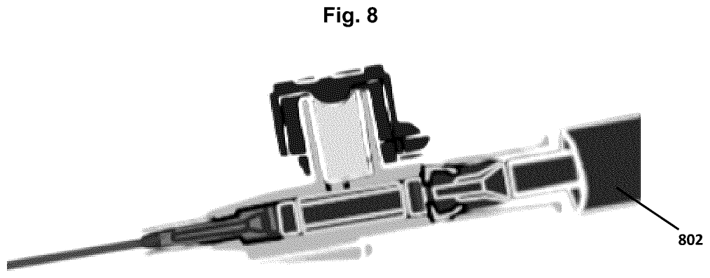

An interplay between the fluid control valve 120 and the actuator 124 is central to the performance of the catheter assembly 100. When the needle 122 or the luer connector 802 (shown in FIG. 8) is inserted, the actuator 124 advances forward, its tapered distal tip gently pressing against the plus-shaped slit 702 and spreading it open. This allows fluid communication for infusion or sampling. When the needle 122 or the luer connector is removed, the actuator 124 retracts passively or remains in place, allowing the slit 702 to elastically return to its closed state. In an embodiments, the plus-shaped slit 702 has a preloaded configuration which ensures hydrostatic sealing even in the presence of back pressure from a patient's vasculature.

In an embodiment, a spring-biased actuator 124 may be employed, where removal of the luer connector or the needle 122 causes the actuator 124 to recoil slightly, reducing prolonged pressure on the slit 702 and improving recovery of the elastic seal.

To further improve this interaction, in some embodiments, the catheter adapter 102 may include internal wings or protrusions (not shown) that define a maximum travel limit for the actuator 124. These act as physical stops, preventing the actuator 124 from damaging the valve 120 by excessive forward movement. This is particularly useful in cases where users apply additional torque or force during connector engagement.

In some embodiments, the actuator 124 may include a locking flange that nests within a groove formed at the periphery of the valve 120. This interlocking interface prevents unintentional actuator migration, especially when the catheter assembly 100 is exposed to vibration or movement during transportation or use.

A few embodiments of the catheter assembly 100 are further presented below without limitations.

In an embodiment, the catheter assembly 100 may be provided such that the predefined angle may range from 2% to 10% such that it facilitates engagement with the luer lock connector and enables forward movement of the actuator 124 to open the fluid control valve 120 and substantially prevent fluid leakage when the needle 122 is removed.

In an embodiment, the catheter assembly 100 may further include the luer lock connector at the proximal end 104 of the catheter adapter 102. The luer lock connector may be adapted to engage with a corresponding luer device through a threaded coupling for providing a secure, leak-resistant connection during fluid administration.

In an embodiment, the plus-shaped slit 702 may include the four intersecting slit segments that may be arranged orthogonally in a perpendicular configuration to form a symmetric cross shape. The four intersecting slit segments may be dimensioned to expand elastically upon insertion of the needle 122 and return to the sealed state upon the removal of the needle 122.

In an embodiment, the plus-shaped slit 702 may include four angled slit segments arranged in a non-perpendicular configuration, such that the slit segments may diverge radially from a central point in a symmetric or asymmetric pattern, wherein the geometry is configured to enhance sealing performance during needle insertion and withdrawal.

In an embodiment, each of the four intersecting slit segments of the plus-shaped slit has similar width.

In an embodiment, each of the four intersecting slit segments of the plus-shaped slit 702 has a variable width such that a central region of the plus-shaped slit 702 may be narrower compared to outer regions of the slit 702, for improving fluid sealing and controlled expansion when the needle is inserted or removed.

In an embodiment, the four intersecting slit segments of the plus-shaped slit 702 may be oriented at an oblique angle relative to a central axis of the fluid control valve 120, to increase sealing pressure when the needle 122 is removed and reduce leakage under varying fluid pressures.

In an embodiment, the catheter adapter 102 may be provided with one or more internal wings configured to restrict forward movement of the actuator 102 beyond a predefined limit. The one or more internal wings may be positioned such that when the luer connector is connected with the actuator 102 and an excessive force is applied, the one or more internal wings may prevent the actuator 124 from excessively advancing into the fluid control valve 120.

In an embodiment, the plus-shaped slit 702 may be configured to vary in width along its length, such that a central portion of the slit 702 may be wider than terminal ends of the slit 702 to facilitate controlled expansion and retraction.

In an embodiment, the plus-shaped slit 702 may be configured to assume similar width along its length.

In an embodiment, the actuator may include a tapered profile, wherein tapering may be adapted to provide a gradual sealing effect as it engages with the plus-shaped slit 702.

In an embodiment, the fluid control valve may include a multi-layered valve structure, with at least one inner elastic sealing layer and an outer reinforcement layer, such that the inner layer may be configured to deform to accommodate needle passage while the outer layer maintains structural integrity to minimize leakage and wear over repeated use. The inner layer and the outer layer may be made of similar or different materials.

In an embodiment, the fluid control valve 120 may include a peripheral groove. The actuator 124 may be configured to interlock with the groove to restrict unintended movement of the actuator 124 with respect to the fluid control valve 120 and thereby control fluid leakage.

In an embodiment, the actuator may include a plurality of micro-ridges along its contact surface. The micro-ridges may be configured to interlock with corresponding micro-textures provided within the plus-shaped slit 702 of the fluid control valve 120, to restrict unintended movement of the actuator 124 with respect to the fluid control valve 120 and thereby control fluid leakage.

In an embodiment, the length of each of the four intersecting slit segments may be same. In another embodiment, the length of one or more of the four intersecting slit segments may be different.

In an embodiment, the catheter assembly 100 described herein may be adapted for use across a range of clinical scenarios. In neonatal or pediatric settings, where fluid volumes are small and pressure sensitivity is high, the valve 120 may be tuned with softer elastomers and narrower slits to reduce required insertion force. For high-flow infusion applications, the valve 120 and the actuator 124 dimensions may be scaled accordingly, ensuring the slit 702 can expand to accommodate larger bore connectors while maintaining sealing performance.

In embodiments, the catheter assembly 100 may be adapted to closed IV catheters, safety IV systems, or integrated pressure monitoring lines, where passive sealing mechanisms may be critical. The plus-shaped valve 120 and the actuator 124 mechanism form a platform technology capable of incorporation across diverse fluid delivery systems.

The catheter assembly 100 may be utilized in clinical settings and constitute a critical component of modern infusion therapy. The catheter assembly 100 may be used in routine IV catheterization and avoid complications, including inadvertent blood spillage. In particular, the catheter assembly 100 may prevent or minimize blood exposure during catheter withdrawal, or upon connection and disconnection with Luer-compatible medical devices.

The causes of such blood exposure have been identified to include, without limitation, backflow of blood from the catheter hub, inadvertent blood splashes, and inadequate compression of the vein during handling of Luer connectors. Consequently, healthcare professionals are routinely exposed to blood and bodily fluids, which may increase the risk of transmission of bloodborne pathogens such as human immunodeficiency virus (HIV), hepatitis B virus (HBV), and hepatitis C virus (HCV). The catheter assembly 100 discussed herein facilitates in avoiding or minimizing such issues.

In this context, the present catheter assembly 100 provides a mechanism to limit or eliminate blood leakage.

The valve 120 discussed herein may be operable multiple times, thereby ensuring protection against blood exposure throughout various stages of catheter use.

The catheter assembly 100 discussed herein helps in reduction or elimination of the need for manual vein compression during catheter placement, thereby minimizing patient discomfort and associated pain.

Substantial reduction in blood exposure after needle removal improves clinician safety.

Decrease in the time required for intravenous catheter insertion due to simplified handling and reduced clean-up helps in medical use.

The catheter assembly 100 helps in minimization of environmental contamination, including contamination of skin, gloves, dressings, clothing, and surrounding surfaces.

The catheter assembly 100 helps in improvement in the psychological experience of patients, particularly those who may become anxious upon viewing blood.

The catheter assembly 100 helps in reusability of the valve 120 function across multiple connections and disconnections during the IV therapy process.

The present catheter assembly 100 with integrated blood control features such as the valve 120 and the actuator 124 is intended for use wherever venous access is required. It may be substituted for conventional IV cannulae in substantially all clinical scenarios involving peripheral IV catheterization. Applications include, but are not limited to, hospitals, emergency care settings, outpatient infusion centers, home care environments, and mobile medical units.

The disclosed catheter assembly 100 may thus provide a safer, more efficient alternative to conventional IV catheters, with added advantages for both healthcare providers and patients.

What is claimed is:

1. A catheter assembly for controlling fluid leakage, the catheter assembly comprising:
a catheter adapter having a distal end, a proximal end, a body portion extending between the distal end and the proximal end, and a lumen defined within the catheter adapter, the lumen extending through the body portion between the distal end and the proximal end;
a catheter extending distally from the distal end of the catheter adapter, the catheter being in fluid communication with the lumen;
a fluid control valve positioned within the lumen of the catheter adapter, the fluid control valve comprising a plus-shaped slit adapted to selectively allow passage of a needle therethrough; and
an actuator deployed within the catheter hub and having a generally circular cross-sectional profile, the actuator comprising a proximal end and a distal end, wherein the distal end is sized to engage with the plus-shaped slit of the fluid control valve and the proximal end has a larger diameter than the distal end, the actuator tapering from the distal end to the proximal end at a predefined angle.

2. The catheter assembly of claim 1, wherein the predefined angle ranges from about 2% to 10%, the angle being configured to facilitate engagement with a luer lock connector and to permit distal movement of the actuator sufficient to open the fluid control valve and reduce fluid leakage upon removal of the needle.

3. The catheter assembly of claim 1, wherein the catheter assembly further comprising a luer lock connector at the proximal end of the catheter adapter, the luer lock connector being adapted to engage with a corresponding luer device through a threaded coupling for providing a secure, leak-resistant connection during fluid administration.

4. The catheter assembly of claim 1, wherein the fluid control valve is retained within an annular groove of the catheter adapter so as to restrict axial movement of the fluid control valve in a proximal direction, a distal direction, or both.

5. The catheter assembly of claim 1, wherein the plus-shaped slit cooperates with the actuator to form a dynamic seal configured to minimize fluid exposure upon withdrawal of the needle.

6. The catheter assembly of claim 1, wherein the needle comprises a beveled distal tip, the beveled distal tip being adapted to facilitate smooth penetration through the plus-shaped slit of the fluid control valve, minimizing deformation of the slit upon removal of the needle.

7. The catheter assembly of claim 1, wherein the plus-shaped slit comprises four intersecting slit segments arranged to define a pre-defined configuration.

8. The catheter assembly of claim 7, wherein the plus-shaped slit comprises the four intersecting slit segments arranged orthogonally in a perpendicular configuration to form a symmetric cross shape, the four intersecting slit segments being dimensioned to expand elastically upon insertion of the needle and return to a sealed state upon the removal of the needle.

9. The catheter assembly of claim 7, wherein the plus-shaped slit comprises the four angled slit segments arranged in a non-perpendicular configuration, such that the slit segments diverge radially from a central point in a symmetric or asymmetric pattern, the geometry being configured to enhance sealing performance during needle insertion and withdrawal.

10. The catheter assembly of claim 1, wherein the catheter adapter is provided with one or more internal wings configured to restrict forward movement of the actuator beyond a predefined limit, such that when a luer connector is connected with the actuator and an excessive force is applied, the one or more internal wings prevent the actuator from excessively advancing into the fluid control valve.

11. The catheter assembly of claim 1, wherein the actuator includes a tapered profile.

12. The catheter assembly of claim 1, wherein the fluid control valve comprises a peripheral groove, the actuator being configured to interlock with the groove to restrict unintended movement of the actuator with respect to the fluid control valve and thereby control fluid leakage.

13. A catheter assembly for controlling fluid leakage, the catheter assembly comprising:
a catheter adapter having a distal end, a proximal end, a body portion extending between the distal end and the proximal end, and a lumen extending through the body portion;
a catheter extending distally from the distal end of the catheter adapter and in fluid communication with the lumen;
a fluid control valve disposed within the lumen of the catheter adapter, the fluid control valve comprising a plus-shaped slit configured to selectively permit passage of a needle therethrough; and
an actuator positioned within the catheter adapter and having a proximal end and a distal end, the distal end being dimensioned to engage the plus-shaped slit of the fluid control valve, the proximal end having a diameter larger than the distal end, the actuator tapering from the distal end toward the proximal end at a predefined angle,
wherein engagement of a luer connector with the actuator urges the actuator distally to open the fluid control valve and reduce fluid leakage following withdrawal of the needle.

14. The catheter assembly of claim 13, wherein the fluid control valve is retained within an annular groove of the catheter adapter to restrict axial movement of the valve.

15. The catheter assembly of claim 13, wherein the plus-shaped slit and the actuator cooperate to form a dynamic seal configured to minimize fluid exposure after removal of the needle.

16. The catheter assembly of claim 13, wherein the needle includes a beveled distal tip configured to pass through the plus-shaped slit of the fluid control valve with reduced deformation of the slit.

17. The catheter assembly of claim 13, wherein the plus-shaped slit comprises four intersecting slit segments arranged to define a cross-shaped configuration.

18. The catheter assembly of claim 17, wherein the four intersecting slit segments are arranged orthogonally to form a perpendicular cross, the slit segments being dimensioned to elastically expand upon insertion of the needle and return to a sealed state upon removal of the needle.

19. A catheter assembly for fluid delivery, the catheter assembly comprising:
a catheter adapter defining a lumen extending between a proximal end and a distal end;
a catheter extending from the distal end of the catheter adapter and in fluid communication with the lumen;
a valve disposed within the lumen and comprising a slit adapted to permit passage of a needle and to substantially reseal following removal of the needle; and
an actuator disposed within the catheter adapter and engageable with the slit of the valve to facilitate controlled opening and closing of the valve during coupling with a connector.

20. The catheter assembly of claim 19, wherein the slit of the valve comprises a plus-shaped configuration formed by four intersecting slit segments arranged to expand elastically upon insertion of the needle and return to a sealed state upon removal of the needle.
